(19) United States
(12) Patent Application Publication
(10) Pub. No.: US 2021/0059945 A1
(43) Pub. Date: Mar. 4, 2021

(54) Oral Drug Delivery System and Method for Fabricating Thereof

(71) Applicant: National Tsing Hua University, Hsinchu (TW)

(72) Inventors: Hsing-Wen Sung, Hsinchu (TW); Yang-Bao Miao, Hsinchu (TW); Kuan-Hung Chen, Hsinchu (TW)

(21) Appl. No.: 16/778,794

(22) Filed: Jan. 31, 2020

(30) Foreign Application Priority Data
Aug. 29, 2019 (TW) 108131118

(51) Int. Cl. A61K 9/16; A61K 31/713; A61K 31/711

(52) U.S. Cl. CPC A61K 9/167; A61K 9/1611; A61K 31/711; A61K 31/713; A61K 9/1652

(57) ABSTRACT

An oral drug delivery system includes a biomimetic mineralized carrier and a yeast capsule. The biomimetic mineralized carrier has a surface with positive charge and includes a metal organic framework having an internal space and a biological macromolecule encapsulated in the internal space of the metal organic framework. A surface of the metal organic framework has a plurality of pores. The yeast capsule is composed of a β-glucan cell-wall shell that removes a cytoplasm from yeast and has a surface with negative charge. The biomimetic mineralized carrier is loaded into the yeast capsule by an electrostatic force to form the oral drug delivery system.

### CROSS REFERENCE TO RELATED APPLICATION

This application claims priority to Taiwanese Patent Application No. 108131118 filed Aug. 29, 2019, the disclosure of which is hereby incorporated by reference in its entirety.

### BACKGROUND

### Technical Field

The present disclosure relates to a drug delivery system and a method fabricating thereof. More particularly, the present disclosure relates to an oral drug delivery system and a method fabricating thereof.

### Description of Related Art

Drugs are substances that have therapeutic effects for curing diseases, reducing suffering of patients, or preventing human diseases. Drugs include natural ingredients, chemically synthesized substances, and biological agents. The general modes of administration include injection administrations (such as intravenous injection, intramuscular injection or subcutaneous injection, etc.), oral administrations (such as oral administration through the gastrointestinal tract, sublingual tablets and oral tablets, etc.) and external administrations (such as transdermal mucosal medication, transdermal absorption medication, transnasal mucosa or pulmonary respiratory tract medication, etc.).

Oral administration is to swallow the drug through the gastrointestinal mucosa and transport it to various parts of the body through the bloodstream to make it function in the body. Oral administration eliminates the need for needles and is convenient to use, which is conducive to patient self-management, so it is considered a promising way of administration. In addition, the gastrointestinal tract has mucosal immune response (secreted immunoglobulin A, S-IgA) and systemic immune response (serum immunoglobulin G, IgG). Therefore, if the drug delivered is a vaccine, a complete immune response can be induced by the oral administration.

However, there are still problems with oral administration. For example, if the active ingredient of an oral drug is a protein, a change in pH in the gastrointestinal (GI) tract may denature it or be degraded by a gastrointestinal protease. Oral administration is also prone to encounter the mucosal barrier formed by tightly arranged epithelial cells in the intestine, reducing its effectiveness. In addition, a prerequisite for a potent immune response to be taken orally is the effective uptake of the vaccine formulation on the mucosa. Therefore, how to develop a new type of oral drug delivery system that can protect the biological macromolecules encapsulated therein resisting the GI conditions and can effectively deliver the biological macromolecules to the body's target has become important development goals in the field of pharmacy today.

### SUMMARY

According to one aspect of the present disclosure, an oral drug delivery system is provided. The oral drug delivery system includes a biomimetic mineralized carrier and a yeast capsule. The biomimetic mineralized carrier has a surface with a positive charge and includes a metal organic framework having an internal space and a biological macromolecule encapsulated in the internal space of the metal organic framework. A surface of the metal organic framework has a plurality of pores. The yeast capsule is composed of a β-glucan cell-wall shell that removes a cytoplasm from a yeast and has a surface with a negative charge. The biomimetic mineralized carrier is loaded into the yeast capsule by an electrostatic force.

According to another aspect of the present disclosure, a method for fabricating the abovementioned oral drug delivery system includes steps as follows. A mixture is provided, a coating step is performed, the biomimetic mineralized carrier is collected, a first solution is provided, a second solution is provided and a loading step is performed. The mixture includes an organic ligand, a metal ion, the biological macromolecule and water. In the coating step, the mixture is subjected to a coordination reaction between the organic ligand and the metal ion in a sonication manner to form the internal space, and the biological macromolecule is in situ encapsulated in the internal space to form the biomimetic mineralized carrier having the surface with the positive charge. The first solution includes the biomimetic mineralized carrier. The second solution includes the yeast capsule composed of the β-glucan cell-wall shell that removes the cytoplasm from the yeast by a chemical method, and the yeast capsule has the surface with the negative charge. In the loading step, the first solution is mixed with the second solution and then shaken for a shaking time, and the biomimetic mineralized carrier is loaded into the yeast capsule by the electrostatic force to form the oral drug delivery system.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiment, with reference made to the accompanying drawings as follows:

FIG. 1A is a structural schematic view showing an oral drug delivery system according to the present disclosure.

FIG. 1B is a structural schematic view showing a biomimetic mineralized carrier according to the present disclosure.

FIG. 1C is a schematic diagram illustrating operation mechanism of the oral drug delivery system of the present disclosure.

FIG. 2 is a flow chart showing a method for fabricating the oral drug delivery system according to the present disclosure.

FIG. 3A is a transmission electron microscope micrograph of a biomimetic mineralized carrier according to Example 1 of the present disclosure.

FIG. 3B is an elemental line-scan profile of the biomimetic mineralized carrier according to Example 1 of the present disclosure.

FIG. 3C is a PXRD pattern of the biomimetic mineralized carrier according to Example 1 of the present disclosure.

FIG. 3D is a distribution of pore sizes in the biomimetic mineralized carrier according to Example 1 of the present disclosure.

FIG. 3E shows an analytical result of enzymatic activity of β-Gal of a biomimetic mineralized carrier according to Example 2 of the present disclosure.

FIGS. 3F, 3G and 3H show analytical results of stability of the biomimetic mineralized carrier in simulated gastrointestinal tract condition according to Example 1 of the present disclosure.

FIG. 3I shows changes in particle size and OVA release profile of the biomimetic mineralized carrier according to Example 1 of the present disclosure.

FIG. 4 is an ultraviolet and visible spectrum of a biomimetic mineralized carrier according to Example 3 of the present disclosure.

FIG. 5A shows scanning electron microscope micrographs and transmission electron microscope micrographs of an oral drug delivery system according to Example 4 of the present disclosure.

FIG. 5B shows analytical results of zeta potential of the oral drug delivery system according to Example 4 of the present disclosure.

FIG. 5C shows analytical results of cytotoxicity of the oral drug delivery system according to Example 4 of the present disclosure.

FIG. 5D shows confocal laser scanning microscope images of the oral drug delivery system according to Example 4 of the present disclosure.

FIGS. 6A and 6B show analytical results of macrophages phagocytosing the oral drug delivery system of Example 4 of the present disclosure.

FIGS. 7A, 7B, 7C, 7D, 7E and 7F show analytical results of in vivo transport route of the oral drug delivery system of Example 4 of the present disclosure.

FIG. 7G shows analytical results of concentration of OVA-specific S-IgA antibodies and IgG antibodies in test animals after administering with the oral drug delivery system of Example 4 of the present disclosure under various dosing regimens.

FIG. 7H shows analytical results of concentration of OVA-specific S-IgA antibodies and IgG antibodies in the test animals after administering with free OVA, OVA@AI-MOFs and the oral drug delivery system of Example 4 of the present disclosure using a three-dose oral immunization schedule.

FIG. 7I shows histological photomicrographs of intestinal villi and liver sections of the test animals.

FIG. 7J shows analytical results of AST and ALT enzyme levels in plasma of the test animals.

FIG. 7K shows histological photomicrographs of stomach, heart, lung, spleen and kidney sections of the test animals.

FIG. 8 shows analytical results of in vivo imaging system of the brain, heart, lungs, liver, spleen, pancreas and kidney after administration with the oral drug delivery system of the present disclosure.

FIG. 9 shows confocal laser scanning microscope images of a brain tissue of the test animal administered with the oral drug delivery system of the present disclosure.

FIG. 10 shows analytical results of immunofluorescence staining on the brain tissue of the test animal administered with the oral drug delivery system of the present disclosure.

### DETAILED DESCRIPTION

The following descriptions of particular embodiments and examples are provided by way of illustration and not by way of limitation. Those skilled in the art will readily recognize a variety of noncritical parameters that could be changed or modified to yield essentially similar results.

Unless otherwise stated, the meanings of the scientific and technical terms used in the specification are the same as those of ordinary skill in the art. Furthermore, the nouns used in this specification are intended to cover the singular and plural terms of the term unless otherwise specified.

The term “individual” or “patient” refers to an animal that is capable of administering an oral drug delivery system of the present disclosure. Preferably, the animal is a mammal.

The term “about” means that the actual value falls within the acceptable standard error of the average, as determined by person having ordinary skill in the art. The scope, number, numerical values, and percentages used herein are modified by the term “about” unless example or otherwise stated. Therefore, unless otherwise indicated, the numerical values or parameters disclosed in the specification and the claims are approximate values and can be adjusted according to requirements.

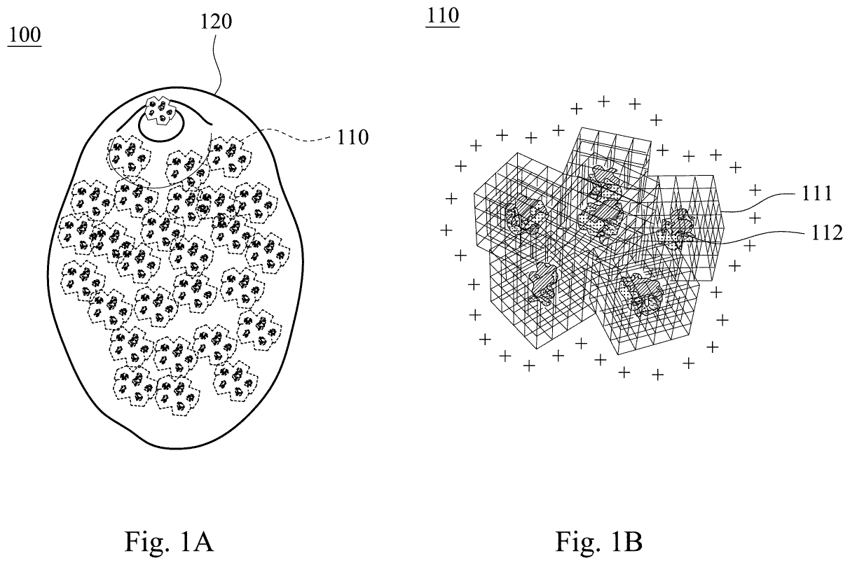

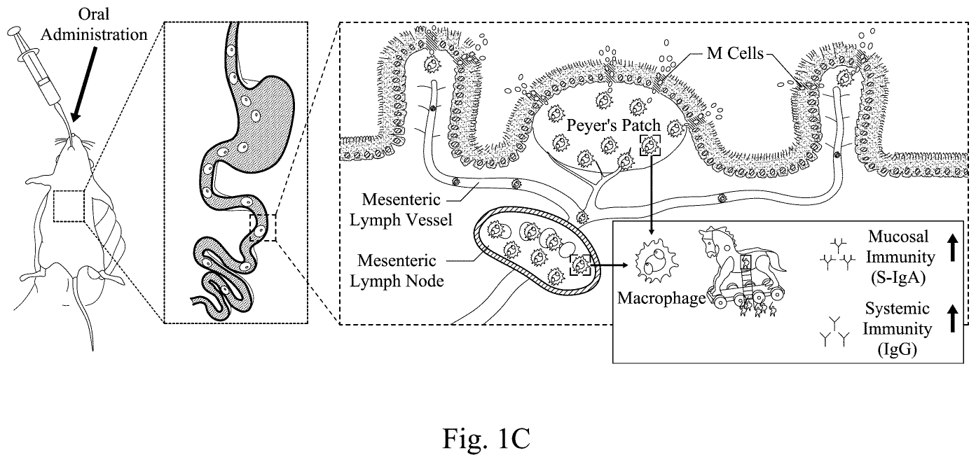

Please refer to FIGS. 1A and 1B. FIG. 1A is a structural schematic view showing an oral drug delivery system 100 according to the present disclosure, and FIG. 1B is a structural schematic view showing a biomimetic mineralized carrier 110 according to the present disclosure. The oral drug delivery system 100 includes the biomimetic mineralized carrier 110 and a yeast capsule 120.

The biomimetic mineralized carrier 110 includes a metal organic framework 111 and a biological macromolecule 112. In particular, the metal organic framework 111 has an internal space, and a surface of the metal organic framework 111 has a plurality of pores. The biological macromolecule 112 is encapsulated in the internal space of the metal organic framework 111 to form the biomimetic mineralized carrier 110 having a surface with a positive charge. A particle size of the biomimetic mineralized carrier 110 can range from 25 nm to 100 nm. Further, the metal organic framework 111 can be MIL-53 (Al, Fe, Cr), MIL-100 (Al, Fe, Cr), MIL-101 (Al, Fe, Cr), MIL-127 (Al, Fe, Cr), PCN-88 (Cu), NU-1000 (Zr) or UIO-66 (Zr). The biological macromolecule 112 can be a nucleic acid or a protein, wherein the nucleic acid can be selected from the group consisting of an oligo-double-stranded DNA, a poly-double-stranded DNA, an oligo-single-stranded DNA, a poly-single-stranded DNA, an oligo-single-stranded RNA and a poly-single-stranded RNA.

The yeast capsule 120 is composed of a β-glucan cell-wall shell that removes a cytoplasm from a yeast. The yeast capsule 120 has a surface with a negative charge, and the biomimetic mineralized carrier 110 is loaded into the yeast capsule 120 by an electrostatic force to form the oral drug delivery system 100. Further, the yeast can be Saccharomyces cerevisiae, Candida albicans, Rhodotorula rubra or Torulopsis utilis.

Therefore, the oral drug delivery system 100 of the present disclosure can protect the biological macromolecules 112 encapsulated therein by biomimetically mineralized metal organic framework 111 for resisting highly acidic and degradative gastrointestinal (GI) conditions and keeping the activity of the biological macromolecules 112 encapsulated therein, and can act synergistically as a delivery vehicle and an adjuvant. The yeast capsule 120 loaded with the biomimetic mineralized carrier 110 can target microfold (M) cells in the intestinal tract, increasing transepithelial absorption of the oral drug delivery system 100, followed by subsequent endocytosis in local macrophages, ultimately accumulating in the mesenteric lymph nodes, and yielding long-lasting immune response. Please refer to FIG. 1C, which is a schematic diagram illustrating operation mechanism of the oral drug delivery system 100 of the present disclosure. Following the oral administration of the oral drug delivery system 100 of the present disclosure, biomimetic exoskeletons of the metal organic framework 111 in the form of armor efficiently protect their encapsulated biological macromolecules 112 against the harsh GI conditions. Concurrently, the yeast capsule 120 act as “Trojan Horses”, carrying the biomimetic mineralized carrier 110 to target M cells and conveying the biological macromolecules 112/adjuvant (the metal organic framework 111) together across the tightly packed mucosal epithelium into the inductive sites of gut-associated lymphoid tissues (GALTs). Following subsequent endocytosis in local antigen-presenting cells (APCs) such as macrophages, the oral drug delivery system 100 ultimately accumulates in the mesenteric lymph nodes (MLNs), activating antigen-specific mucosal S-IgA and serum IgG responses.

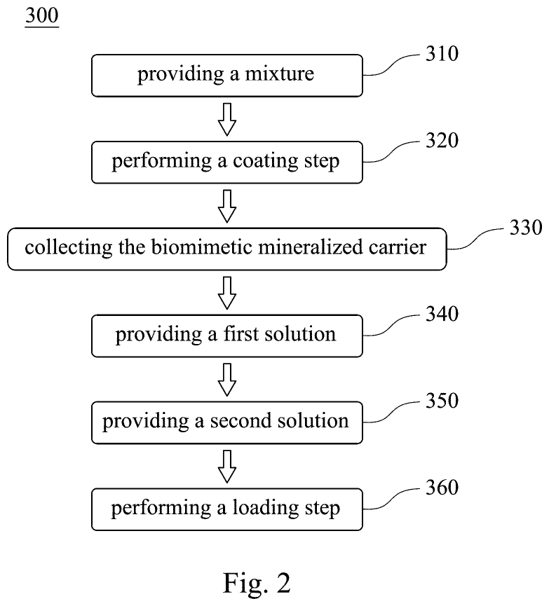

Please refer to FIG. 2, which is a flow chart showing a method for fabricating the oral drug delivery system 300 according to the present disclosure. The method for fabricating the oral drug delivery system 300 includes Step 310, Step 320, Step 330, Step 340, Step 350 and Step 360.

In Step 310, a mixture is provided. The mixture includes an organic ligand, a metal ion, the biological macromolecule and water. A concentration ratio of the organic ligand, the metal ion and the biological macromolecule in the mixture can be 1:1:0.004 to 1:1:0.018. The organic ligand can be 2-amino terephthalic acid, terephthalic acid, 3,3′-(naphthalene-2,7-diyl) dibenzoic acid, 3,3′,5,5′-azobenzenetetracarboxylic acid or biphenyl-4,4′-dicarboxylic acid. The metal ion is formed by dissolving a metal salt in hydrolysis, and the metal salt can be AlCl₃, Al₂(SO₄)₃, Al(NO₃)₃, aluminium isopropoxide, FeCl₃, Fe₂(SO₄)₃, Fe(NO₃)₃, CuCl₂, CuSO₄, Cu(NO₃)₂, ZrCl₄, Zr(NO₃)₄, Zr(SO₄)₂, CrCl₃, Cr(NO₃)₃ or zirconium citrate. The biological macromolecule is a nucleic acid or a protein, wherein the nucleic acid can be selected from the group consisting of an oligo-double-stranded DNA, a poly-double-stranded DNA, an oligo-single-stranded DNA, a poly-single-stranded DNA, an oligo-single-stranded RNA and a poly-single-stranded RNA.

In Step 320, a coating step is performed. The mixture is subjected to a coordination reaction between the organic ligand and the metal ion in a sonication manner to form the internal space, and the biological macromolecule is in situ encapsulated in the internal space to form the biomimetic mineralized carrier having the surface with the positive charge. The sonication manner can be to process the mixture using a sonicator at 30% to 50% amplitude at 0° C. for 90 to 150 minutes.

In Step 330, the biomimetic mineralized carrier is collected, which can be achieved through steps such as reduced pressure concentration, centrifugation, filtration, washing or drying.

In Step 340, a first solution is provided, wherein the first solution includes the biomimetic mineralized carrier obtained through Steps 310 to 330.

In Step 350, a second solution is provided, wherein the second solution includes the yeast capsule. The yeast capsule is composed of the β-glucan cell-wall shell that removes the cytoplasm from the yeast by a chemical method, and the yeast capsule has the surface with the negative charge. For example, the yeast is treated by alkali, acid, and organic solvents to obtain the β-glucan cell-wall shell to prepare the yeast capsule. Preferably, the yeast can be destroyed by acid and alkali, and then its cytoplasm can be removed by isopropyl alcohol and acetone solution. The yeast can be Saccharomyces cerevisiae, Candida albicans, Rhodotorula rubra or Torulopsis utilis.

In Step 360, a loading step is performed. The first solution is mixed with the second solution and then shaken for a shaking time, and the biomimetic mineralized carrier is loaded into the yeast capsule by the electrostatic force to form the oral drug delivery system. The shaking time can be 2 to 6 hours. A weight ratio of the biomimetic mineralized carrier in the first solution and the yeast capsule in the second solution can be 1:1 to 2:1.

Therefore, the method for fabricating the oral drug delivery system is a simple one-pot method for fabricating the biomimetic mineralized carrier. The organic ligand and the metal ion are processed by mild ultrasound to synthesize a nanoscale metal organic framework, and further mimic the secretion of inorganic minerals by living organisms to form exoskeletons to encapsulate the biological macromolecule in the metal organic framework to form the biomimetic mineralized carrier with the positive charge on the surface. Furthermore, the biomimetic mineralized carrier is loaded into the yeast capsule with the negative charge on the surface by electrostatic force to form the oral drug delivery system.

The oral drug delivery system has been described as mentioned above. In the following, reference will now be made in detail to the present embodiments of the present disclosure, examples and comparative examples of which are illustrated in the accompanying drawings. The accompanied effects of the oral drug delivery system in the examples and comparative examples for demonstrating the effect of the oral drug delivery system. However, the present disclosure is not limited thereto.

### EXAMPLES

### I. Biomimetic Mineralized Carrier of the Present Disclosure and Fabrication Method Thereof

### 1st Embodiment

### 1.1. Fabrication, Structure and Characteristic Analysis of Biomimetic Mineralized Carrier

To test the optimal preparation condition, the biomimetic mineralized carrier of Example 1 is fabricated in this experiment first. The morphology of the biomimetic mineralized carrier of Example 1 is observed by transmission electron microscope (TEM, JEM-2100F, JEOL Technics), and its particle size and surface charge are measured by dynamic light scattering (DLS, Zetasizer, 3000 HS, Malvern Instruments, Worcestershire). The crystalline structure of the biomimetic mineralized carrier of Example 1 is determined using an X-ray diffractometer (Cu Kα radiation, XRD-6000, Shimadzu), and its pore size is analyzed by the BJH method (BELSORP-mini, BEL).

The organic ligand used in the biomimetic mineralized carrier of Example 1 is 2-amino terephthalic acid, the metal salt is aluminum isopropoxide, the biological macromolecule is a protein, in which ovalbumin (OVA) is used as an example, and the detailed preparation process is as follows. 0.5 mmol of aluminum isopropoxide, 0.5 mmol of 2-amino terephthalic acid, and 3×10⁻³ mmol of OVA are dissolved in 30 mL of deionized (DI) water to obtain the mixture and then vortexed for 60 seconds at room temperature. The mixture is mildly sonicated using a VCX 750 sonicator (Sonics & Materials, Newtown, Conn., USA) at 40% amplitude at 0° C. for 120 minutes. The obtained biomimetic mineralized carriers of Example 1 (hereafter referred to as OVA@AI-MOFs) are centrifuged at 18,000 rpm for 30 minutes, washed twice using DI water, and then rinsed with an aqueous sodium dodecyl sulfate (SDS) solution (5% by w/w) at 50° C. to remove free OVA from their surfaces.

To quantify the loading content (LC) and the loading efficiency (LE) in the OVA@AI-MOFs, weighed test samples are dissolved in ethylenediaminetetraacetic acid (EDTA, 0.1 M) and then shaken at room temperature for 3 hours to release their encapsulated OVA. The amount of released OVA is quantified using a Pierce™ BCA Protein Assay Kit (Thermo Fisher Scientific, Waltham) and used to calculate the LC and the LE of OVA in the OVA@AI-MOFs using the following equations.

$$\begin{matrix}LC ( \% ) = \frac{weight of OVA in OVA @ AI \text{-} MOFs}{weight of OVA @ AI \text{-} MOFs} \times 100 \% \text{;} & equation I \\ LE ( \% ) = \frac{weight of OVA in OVA @ AI \text{-} MOFs}{total amount of OVA added} \times 100 \% . & equation II\end{matrix}$$

Please refer to Table 1, which shows the LC and the LE of the OVA@AI-MOFs that are synthesized using various feeding concentration ratios of OVA/AI ions/2-aminoterephthalic acid.

TABLE 1

| Feeding concentration | | |
|---|---|---|
| ratio of OVA/Al ions/ | | |
| 2-aminoterephthalic acid | LC (%) | LE (%) |
| 0.004:1.0:1.0 | 9.6 ± 0.9 | 97.6 ± 2.1 |
| 0.007:1.0:1.0 | 14.7 ± 1.3 | 94.1 ± 4.8 |
| 0.014:1.0:1.0 | 12.0 ± 0.5 | 82.3 ± 9.1 |
| 0.018:1.0:1.0 | 8.2 ± 1.1 | 73.2 ± 8.6 |

As shown in Table 1, as the feeding concentration of the OVA increased, the LC of OVA increased, reaching a maximum at the OVA/AI ions/2-aminoterephthalic acid feeding concentration ratio of 0.007:1.0:1.0, which yielded the LC of 14.7±1.3% and the LE of 94.1±4.8% (n=6 batches). Therefore, the biomimetic mineralized carrier is prepared under this formulation in the following experiments.

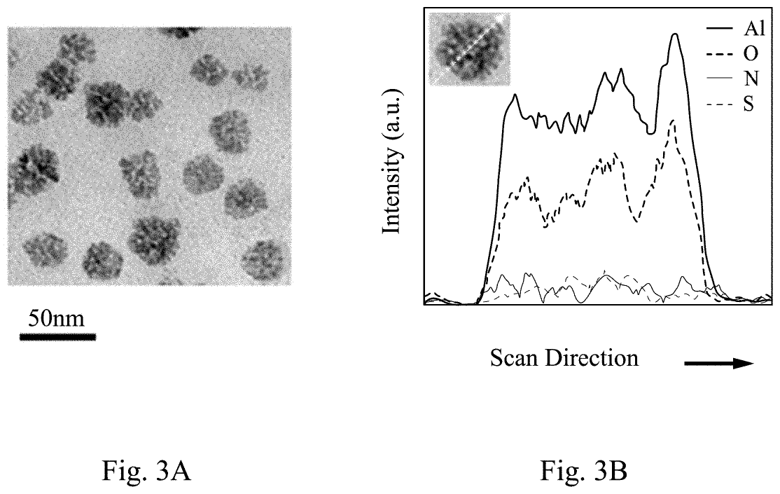

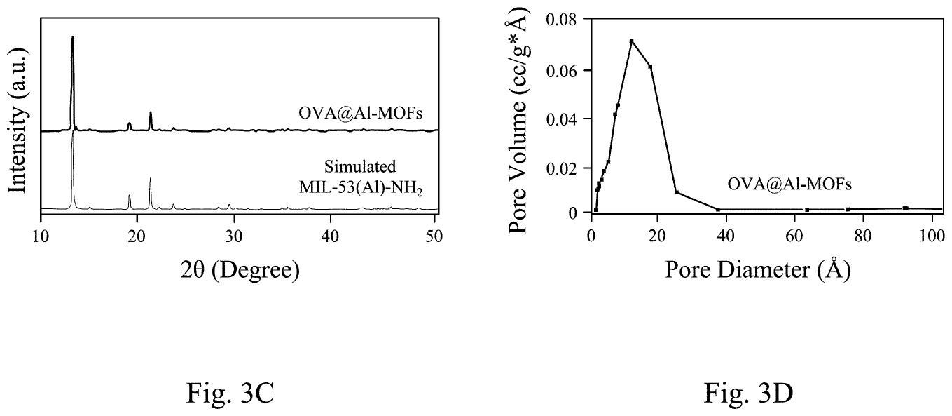

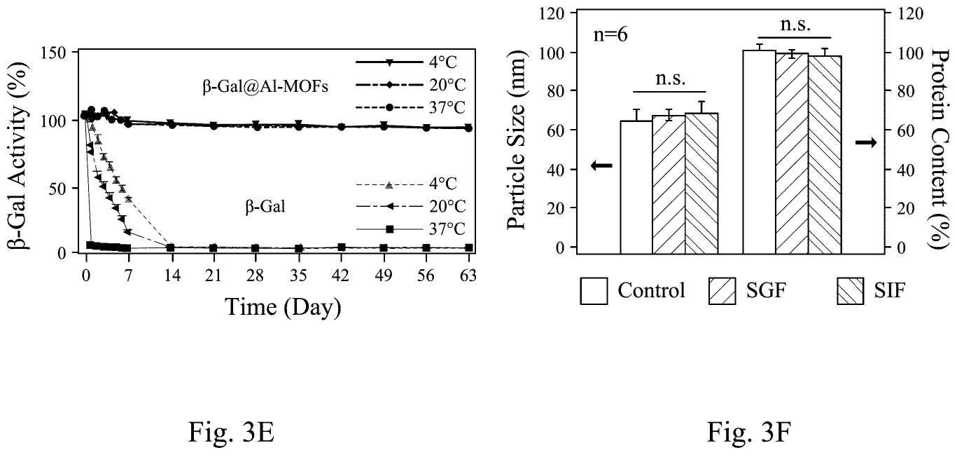

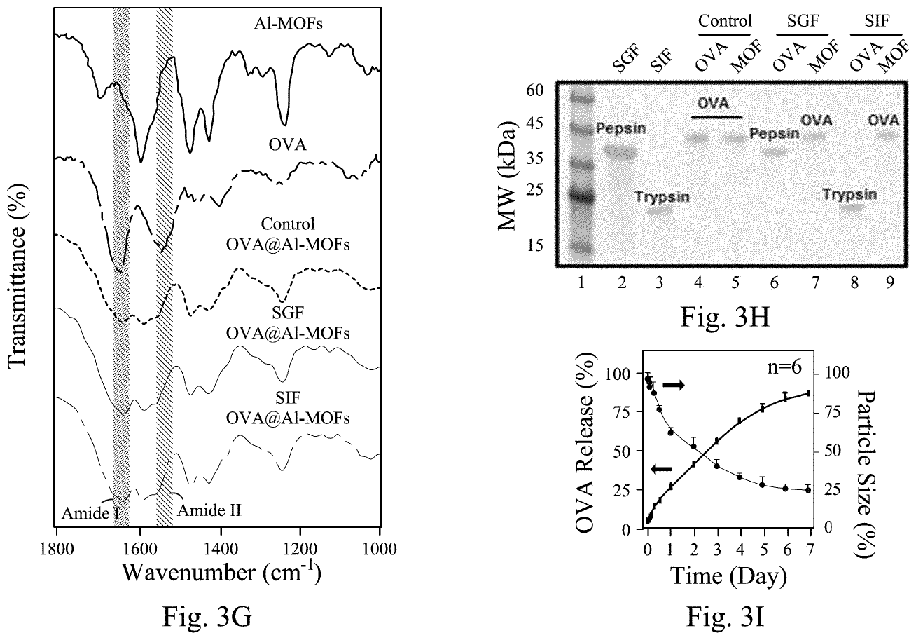

Please refer to FIGS. 3A to 3D, which show analytical results of the biomimetic mineralized carrier according to Example 1 of the present disclosure, wherein FIG. 3A is a transmission electron microscope micrograph, FIG. 3B is an elemental line-scan profile, FIG. 3C is a PXRD pattern, and FIG. 3D is a distribution of pore sizes.

In FIG. 3A, the as-optimized OVA@AI-MOFs have a popcorn-shaped morphology with an average particle size of 65.2±8.9 nm and a zeta potential of 28.7±4.8 mV, as determined by dynamic light scattering (DLS, n=6 batches). In FIG. 3B, analysis of the energy-dispersive X-ray (EDX) spectroscopic line-scan of a TEM sample reveals elemental compositions of Al and O (AI-MOFs) as well as N and S (OVA) in the OVA@AI-MOFs that reflects the successful encapsulation of OVA in the AI-MOFs. The crystalline structure of the OVA@AI-MOFs is investigated by powder X-ray diffraction (PXRD). In FIG. 3C, the PXRD pattern of the experimentally synthesized OVA@AI-MOFs is similar to the simulated pattern of pure MIL-53(Al)—NH₂, suggesting that the encapsulation of OVA does not significantly modify the crystalline structure of AI-MOFs. In FIG. 3D, the diameter of the pore size in the OVA@AI-MOF crystals, as determined by the Barrett-Joyner-Halenda (BJH) method, is ca.15.0±3.0 Å.

### 1.2. Stability of Biomimetic Mineralized Carrier

To determine whether the biomimetic mineralized carrier of the present disclosure can preserve the activity of the protein antigen at various ambient temperatures for long periods, another model enzyme, β-galactosidase (β-gal), is encapsulated in the AI-MOFs as the biomimetic mineralized carrier of Example 2 (hereafter referred to as β-Gal@AI-MOFs), and enzymatic activity of β-Gal in β-Gal@AI-MOFs is further measured to determine the stability of protein activity. The β-Gal@AI-MOFs are stored in normal saline at 4° C., 20° C., or 37° C. for predetermined intervals (0-63 days), before their enzymatic activity is quantified. The enzymatic activity of the β-Gal is evaluated following the manufacturer's instructions (Thermo Fisher Scientific).

Please refer to FIG. 3E, which shows an analytical result of enzymatic activity of β-Gal of the β-Gal@AI-MOFs. FIG. 3E plots the stabilities of free β-gal and the β-Gal@AI-MOFs that had been stored at 4° C., 20° C., or 37° C. for nine weeks. The free enzyme exhibits a substantial loss of activity within two weeks at all tested ambient temperatures, while the β-Gal@AI-MOFs retain ca. 90% of their activity after nine weeks. These experimental results reveal the advantage of the biomimetic mineralized carrier of the present disclosure in long-term preservation of the activity of their armored protein at ambient temperatures, potentially solving the problem of the need to refrigerate distributed vaccines or protein drugs.

Another problem to be solved with oral drugs is their physical stability during transport through the GI tract. An in vitro test is conducted to evaluate the stability of the biomimetic mineralized carrier of the present disclosure under the GI conditions. The OVA@AI-MOFs are incubated individually in simulated gastric fluid (SGF) and simulated intestinal fluid (SIF) at 37° C. The SGF is an HCl solution at pH 2.0 containing NaCl (0.2% by w/v) and pepsin (0.5 mg/mL), and the SIF is an aqueous solution at pH 7.0 containing trypsin (2.5 mg/mL). After predetermined durations, the stability of the incubated OVA@AI-MOFs is obtained by examining their particle size and OVA content. The integrity of OVA that is encapsulated in the OVA@AI-MOFs is analyzed by Fourier-transform infrared (FT-IR) spectroscopy (Perkin-Elmer, Buckinghamshire) and sodium dodecyl sulfate polyacrylamide gel electrophoresis (SDS-PAGE).

Please refer to FIGS. 3F, 3G and 3H, which show analytical results of stability of the OVA@AI-MOFs in simulated GI tract condition, wherein FIG. 3F shows the analytical result of particle size and OVA content of the OVA@AI-MOFs, FIG. 3G shows FT-IR spectra, and FIG. 3H shows SDS-PAGE results.

In FIG. 3F, the particle size and the OVA content of the tested OVA@AI-MOFs that had been treated in SGF or SIF are similar to those detected in an untreated control (P>0.05). Additionally, the results of SDS-PAGE in FIG. 3H indicates that the OVA@AI-MOFs remain intact throughout the course of treatment with SGF or SIF (lanes 7 and 9, respectively), whereas no obvious band is observed for the free OVA under either treatment (lanes 6 and 8, respectively). FT-IR spectroscopy is known to be sensitive to the secondary structure of proteins. The results of the FT-IR spectra in FIG. 3G also demonstrate that the SGF-treatment or SIF-treatment of the OVA@AI-MOFs do not change their structural characteristics (bands in the ranges of 1640-1660 cm⁻¹ and 1510-1560 cm⁻¹ are amide I and II bands, respectively) from those observed in the native OVA. These analytical data reveal that the OVA@AI-MOFs are resistant to simulated gastric and intestinal conditions and imply that the OVA@AI-MOFs are likely to remain intact in the GI tract in vivo.

The results of the examples in this part show that the biomimetic mineralized carrier of the present disclosure can form a protective exoskeleton on the biological macromolecules that acts as armor, providing extraordinary stability during storage in normal saline and under harsh GI conditions. Upon exposure to the ambient temperature or gastric acid, confinement within the metal organic framework prevents protein encapsulated from unfolding, reducing its denaturation. Additionally, the small pore size of the biomimetic mineralized carrier (for example, the OVA@AI-MOFs with ca. 15 Å in diameter) displays an effective barrier that excludes the relatively large GI enzymes (45×49×62 Å³ for pepsin and 49×39×33 Å³ for trypsin), limiting their proteolysis. Accordingly, the biomimetic mineralized carrier of the present disclosure can withstand the acidic conditions of the stomach and survive the protein encapsulated therein in the highly digestive environment of the GI tract.

### 1.3. In Vitro Degradability of Biomimetic Mineralized Carrier

The condition for the release of the biological macromolecule encapsulated in the biomimetic mineralized carrier of the present disclosure after entering the GI tract is evaluated in this experiment. When the biomimetic mineralized carrier is endocytosed in macrophages and exposed to the intracellular fluid, which has a high concentration of phosphate ions, the graduate disintegration of the biomimetic mineralized carrier by the competitive replacement of their organic linkers by phosphate ions is expected, initiating the biological macromolecule release.

Please refer to FIG. 3I, which shows changes in particle size and OVA release profile of the OVA@AI-MOFs. FIG. 3I reveals that the OVA@AI-MOFs in phosphate-buffered saline (PBS), which simulates intracellular fluid, could be slowly disintegrated, reducing their particle size and releasing their encapsulated OVA in a sustained manner over a period of approximately seven days.

### 2nd Embodiment

A biomimetic mineralized carrier of Example 3 is fabricated in this experiment first. The organic ligand used in the biomimetic mineralized carrier of Example 3 is 2-amino terephthalic acid, the metal salt is aluminum isopropoxide, the biological macromolecule is a nucleic acid, in which DNA is used as an example, and the detailed preparation process is as follows. 0.5 mmol of aluminum isopropoxide, 0.5 mmol of 2-amino terephthalic acid, and 100 ng of DNA are dissolved in 30 mL of DI water to obtain the mixture and then vortexed for 60 seconds at room temperature. The mixture is mildly sonicated using the VCX 750 sonicator at 40% amplitude at 0° C. for 120 minutes. The obtained biomimetic mineralized carriers of Example 3 (hereafter referred to as DNA@AI-MOFs) are centrifuged at 18,000 rpm for 30 minutes, washed twice using DI water to remove free DNA from their surfaces. The obtained DNA@AI-MOFs are incubated individually in SGF and SIF at 37° C. to evaluate the stability of the DNA@AI-MOFs under the GI conditions.

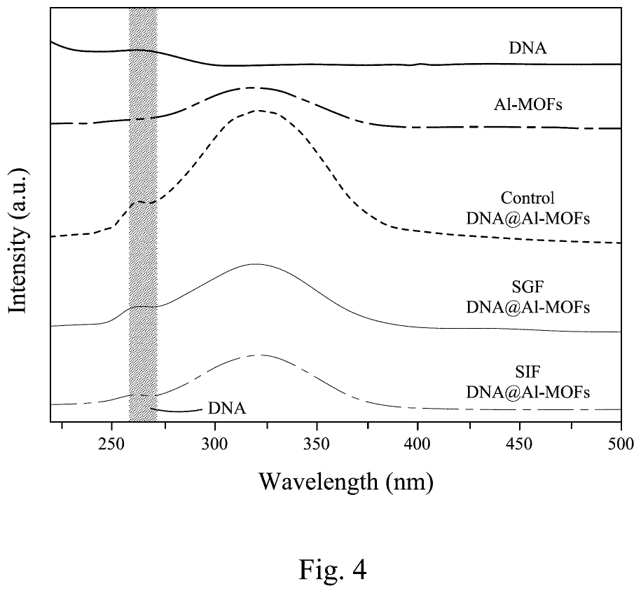

Please refer to FIG. 4, which is an ultraviolet and visible spectrum of the DNA@AI-MOFs. The results of ultraviolet and visible spectrum show that the DNA@AI-MOFs in the control group have an absorption peak at 260 nm, showing that the DNA is encapsulated in the DNA@AI-MOFs. Moreover, under simulated conditions of gastric fluid and intestinal fluid, the absorption peak of DNA@AI-MOFs at 260 nm does not change significantly, which proves that the DNA encapsulated in the DNA@AI-MOFs has not been degraded by strong acids and enzymes of the GI tract.

### II. Oral Drug Delivery System of the Present Disclosure and Fabrication Method Thereof

### 2.1. Fabrication, Structure, Characteristic and Cytotoxicity Analysis of Oral Drug Delivery System

The oral drug delivery system of the present disclosure is further fabricated under the optimal condition described above for fabricating the biomimetic mineralized carrier, and the biomimetic mineralized carrier used in this experiment is the OVA@AI-MOFs. The synthesized OVA@AI-MOFs are dissolved in 10 mL of DI water to obtain a first solution. The yeast capsules (YCs) are further prepared. In this experiment, Saccharomyces cerevisiae is treated with alkali, acid and organic solvents to remove its cytoplasm to obtain the β-glucan cell wall shell, which is the prepared the yeast capsule and can be further freeze-dried and stored for subsequent use. 100 mg of dry YCs are first incubated in 100 mL of DI water for 30 minutes to obtain a second solution. The first solution is mixed with the second solution. Following 4 hours of incubation with shaking, the biomimetic mineralized carrier is loaded into the yeast capsule to form the oral drug delivery system of Example 4 (hereafter referred to as OVA@AI-MOFs/YCs). Then the OVA@AI-MOFs/YCs are collected by centrifugation at 2,500 rpm for 10 minutes. The collected OVA@AI-MOFs/YCs are then thoroughly washed using DI water to remove unloaded OVA@AI-MOFs. The amount of the OVA@AI-MOFs in the YCs is determined by quantifying the unloaded OVA@AI-MOFs in aqueous solution using a BCA Protein Assay Kit (Thermo Fisher Scientific).

To quantify the LC and the LE in the OVA@AI-MOFs/YCs, the OVA@AI-MOFs/YCs are prepared at different feeding weight ratios of the OVA@AI-MOFs and the YCs, and the LC and the LE are further calculated. Please refer to Table 2, which shows the LC and the LE of the OVA@AI-MOFs/YCs prepared with different feeding weight ratios of the OVA@AI-MOFs and the YCs.

TABLE 2

| Feeding weight ratio of | | |
|---|---|---|
| OVA@Al-MOFs/YCs | LC (%) | LE (%) |
| 1.0:1.0 | 5.9 ± 0.5 | 98.2 ± 1.7 |
| 1.5:1.0 | 7.0 ± 0.8 | 91.5 ± 2.6 |
| 2.0:1.0 | 5.6 ± 0.7 | 84.1 ± 6.3 |

As shown in Table 2, when the feeding weight ratio of the OVA@AI-MOFs and the yeast capsules is 1.5:1.0, the LC of OVA@AI-MOFs/YCs reaches the maximum value (7.0±0.8%), and the LE is 91.5±2.6% (n=6 batches). Therefore, the oral drug delivery system is prepared under this formulation in the following experiments.

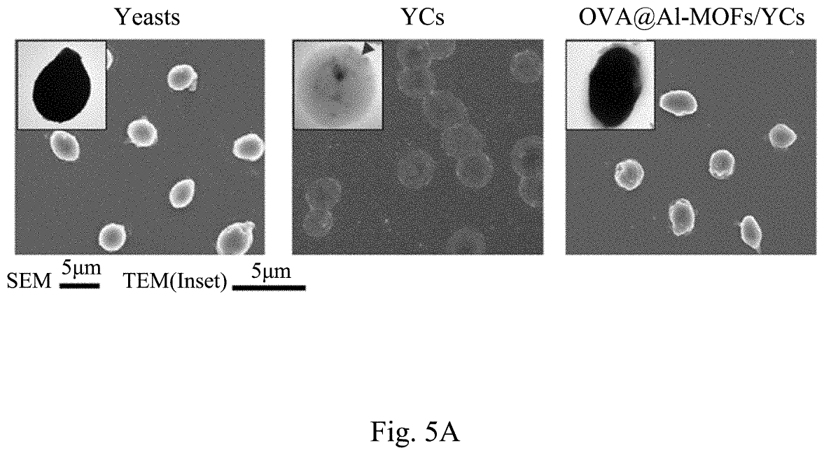

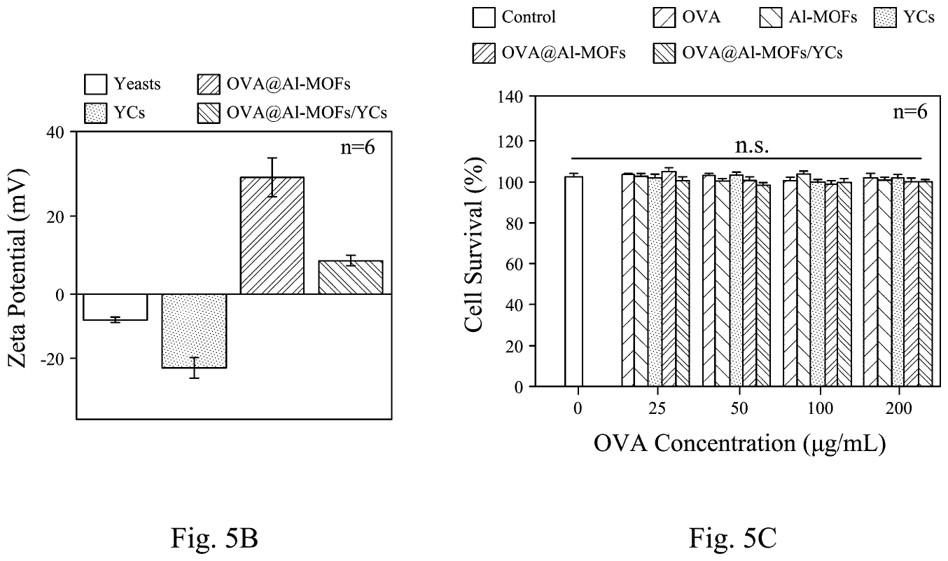

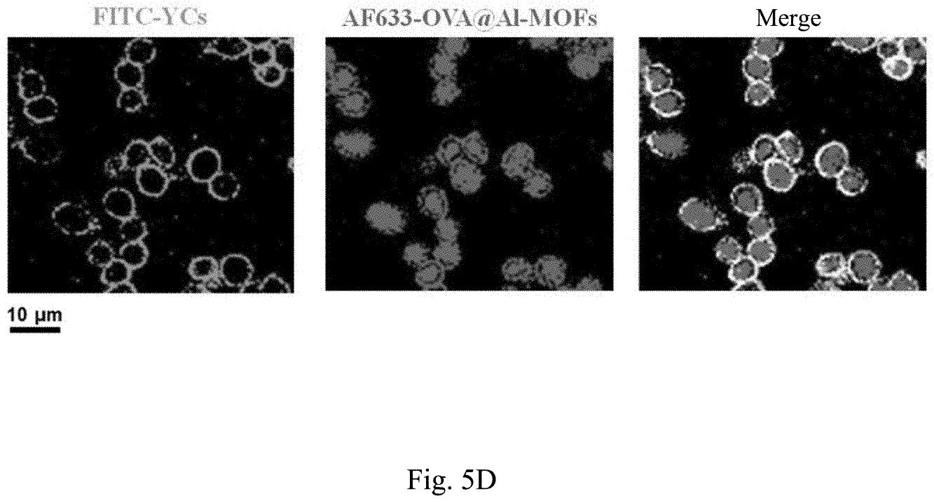

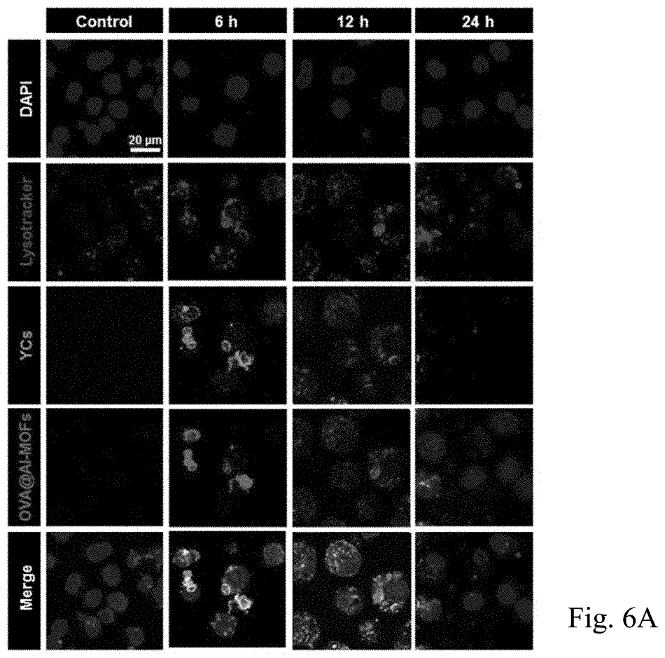

First, the morphology of the as-prepared OVA@AI-MOFs/YCs is determined by scanning electron microscope (SEM, Hitachi SU8010, Hitachi High-Technologies) and TEM (JEM-2100F). Please refer to FIG. 5A, which shows SEM micrographs and TEM micrographs of the OVA@AI-MOFs/YCs, wherein each large image is the SEM micrograph, each small image is the TEM micrograph of the corresponding sample, the left one is Saccharomyces cerevisiae without cytoplasm removal, the middle one is the YCs obtained after treatment, and the right one is the OVA@AI-MOFs/YCs. In FIG. 5A, the untreated yeast is fully rounded and has a diameter of about 3 to 5 μm. Following the removal of their cytoplasm and other cell-wall polysaccharides, hollow β-glucan cell-wall shell with significantly shriveled structures is visible in the SEM micrograph, and the contrast in the TEM micrograph is substantially reduced. Most YCs have one large pore with a diameter of ca. 900 nm (as indicated by the arrowhead in the TEM inset), as a result of the bud scar. In addition, the SEM micrograph reveals that the packed OVA@AI-MOFs/YCs have a densely structure after they had been loaded with the OVA@AI-MOFs.

The zeta potential of the OVA@AI-MOFs/YCs is determined using DLS. Please refer to FIG. 5B, which shows analytical results of zeta potential of the OVA@AI-MOFs/YCs. Zeta potential measurements demonstrate that the YCs are negatively charged, and, after they are packed with the positively-charged OVA@AI-MOFs (28.7±4.8 mV), the zeta potential is changed from −18.3 mV to 8.4 mV.

The cytotoxicities of the OVA@AI-MOFs/YCs at various concentrations of their encapsulated OVA (0-200 μg/mL) and of their components (free OVA, the AI-MOFs, the OVA@AI-MOFs, and the YCs) are studied in vitro by co-culturing with Caco-2 cells. After 24 hours of co-culturing, cell viability is evaluated using a CellTiter-Glo® Luminescent Cell Viability Assay Kit (Promega, Madison, Wis., USA). Human epithelial Caco-2 cell line is a reliable in vitro model for an intestinal cytotoxicity study.

Please refer to FIG. 5C, which shows analytical results of cytotoxicity of the OVA@AI-MOFs/YCs. In FIG. 5C, no significant difference is detected between the cell viability of Caco-2 cells that had been treated with the OVA@AI-MOFs/YCs at any of the test concentrations (P>0.05) and that of untreated control cells. In addition, no significant difference is detected between the cell viability of Caco-2 cells that had been treated with free OVA, the YCs or the AI-MOFs (P>0.05) and that of untreated control cells.

To trace the OVA@AI-MOFs that are packed into the YCs, the YCs and the OVA are fluorescently labeled with FITC (hereafter referred to as FITC-YC) and Alexa Flour 633 (hereafter referred to as AF633-OVA), respectively, and observed using confocal laser scanning microscopy (CLSM, Zeiss LSM780, Carl Zeiss, Jena GmbH). Please refer to FIG. 5D, which shows CLSM images of the OVA@AI-MOFs/YCs. In FIG. 5D, the AF633-OVA@AI-MOFs (pink) are successfully loaded into the FITC-YCs (green).

### 2.2. Uptake of Oral Drug Delivery System by Macrophages and their Maturation

To activate desired immune responses, antigen-presenting cells (APCs) must take up the vaccine. A murine macrophage cell line (RAW264.7), which can recognize the β-glucans of the YCs via its Dectin-1 receptor, is used to evaluate the uptake of as-prepared OVA@AI-MOFs/YCs. RAW264.7 macrophages (1×10⁶ cells/mL) are incubated with AF633-OVA@AI-MOFs/FITC-YCs. Following incubation for predetermined periods (6, 12, and 24 hours), the cells are collected, incubated with a fresh medium that contained LysoTracker™ Red DND-99, thoroughly washed in PBS, stained with DAPI, and then observed using CLSM.

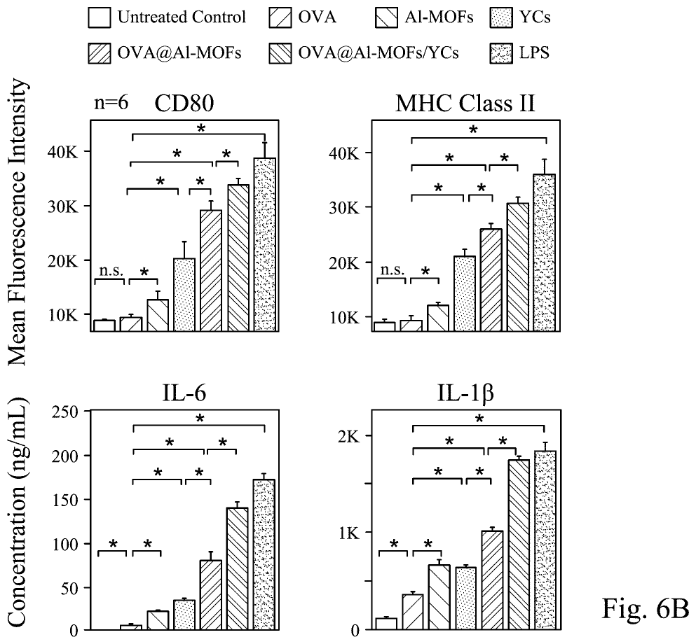

Please refer to FIG. 6A, which shows CLSM images of the RAW264.7 macrophages. In FIG. 6A, at 6 hours of incubation, the intracellular colocalization of green fluorescence (FITC-YCs) and red fluorescence (LysoTracker-stained endo/lysosomes) is clearly visible, indicating that the receptor-targeted uptake of the OVA@AI-MOFs/YCs proceeded via an endo/lysosomal pathway, which is essential for the delivery and processing of antigens. Macrophage cells are known to endocytose large particles with diameters of 1-10 μm. As the incubation time (12 hours) increased, degradation of the endocytosed FITC-YCs, as indicated by the fragmentation of green fluorescence, is detected inside the cells owing to the presence of a wide range of intracellular proteases. A longer incubation time (24 hours) resulted in almost complete degradation of the FITC-YCs; meanwhile, uniform pink fluorescence (AF633-OVA) is observed in the cells. These results demonstrate that upon the enzymatic degradation of the YCs, their loaded OVA@AI-MOFs are directly exposed to the phosphate ion-containing intracellular fluid, causing the disintegration of the AI-MOF armor, triggering the intracellular release of their encapsulated OVA molecules (antigen) and the disintegrated AI ions (adjuvant).

The cellular uptake of vaccine may cause the activation of macrophages, upregulating their surface expressions of co-stimulatory factors (such as CD80 and MHC class II), which are hallmarks of the maturation of macrophages, and promoting the secretion of pro-inflammatory cytokines [such as interleukin 6 (IL-6) and interleukin 1β (IL-1β)], which are important in modulating immune responses. The maturation of macrophages that are separately incubated with the OVA@AI-MOFs/YCs that contained an OVA concentration of 100 μg/mL and their components (free OVA, the AI-MOFs, the OVA@AI-MOFs, and the YCs) is evaluated. In addition, cells that received no treatment or are treated with lipopolysaccharide (LPS), a well-known macrophage maturation agent, are used as an untreated control and a positive control, respectively. Twenty-four hours after incubation, the cells and the culture supernatants in each group are separately harvested. The harvested cells are labeled with APC-conjugated anti-mouse CD80 antibody (eBioscience, San Diego) or Alexa Fluor 647-conjugated anti-mouse MHC class II antibody (BioLegend, San Diego) and then analyzed using a BD Accuri™ C6 flow cytometer (BD Biosciences, San Jose). The concentrations of IL-6 and IL-1β cytokines in the harvested supernatants are obtained by a Cytometric Bead Array (CBA, BD Bioscience).

Please refer to FIG. 6B, which shows the analytical results of macrophages maturation markers. Fluorescence-activated cell sorting (FACS) measurements reveal that the macrophages that are activated by LPS exhibited high concentrations of the maturation markers CD80 and MHC class II and the pro-inflammatory cytokines IL-6 and IL-1β. The OVA@AI-MOFs exhibit considerably higher levels of CD80, MHC class II, IL-6 and IL-1β than free OVA (P<0.05), suggesting that AI-MOFs may function as an effective adjuvant-based antigen delivery system. Notably, stimulation by the YCs alone substantially increases the cellular expression levels of CD80, MHC class II, IL-6, and IL-1β (P<0.05), indicating that the YCs can be used as an adjuvant, consistent with previous reports. As well as providing the inherent adjuvant function of the YCs, the OVA@AI-MOFs/YCs further augment the cellular levels of maturation markers and pro-inflammatory cytokines over those in the group that is treated with the OVA@AI-MOFs (P<0.05). These experimental results demonstrate that the oral drug delivery system of the present disclosure can be endocytosed by macrophages via receptor-targeted uptake, stimulating their maturation and cytokine release, probably enhancing their immunostimulatory activity in vivo.

### 2.3. In Vivo Transport Route of Oral Drug Delivery System

The ability of the oral drug delivery system of the present discloure to improve the transportation of the OVA@AI-MOFs through the intestinal barrier is examined in this experiment. To investigate in vivo transport route of the OVA@AI-MOFs/YCs, test animals are orally treated with fluorescence-labeled OVA@AI-MOFs/YCs as the experimental group. Four hours following treatment, the test animals are euthanized and their GI tracts and intestinal lymphatic systems (villus and Peyer's Node in mesenteric lymph nodes) are retrieved, and the distribution of the FITC-YCs is confirmed using CLSM. The test animals that are treated with the AF633-OVA@AI-MOFs served as a control group. To confirm the lymphatic transport of the OVA@AI-MOFs/YCs, the test animals are orally pretreated with laminarin, which can block agonistic β-glucan binding to Dectin-1. Three hours before the oral administration of the fluorescence-labeled OVA@AI-MOFs/YCs, the test animals are treated with orally administered laminarin, which inhibits the entry of the drug into the lymphatic system, as an experimental control group. The test animals only treated with the laminarin is a negative control group. The doses of the laminarin administered to the experimental control group and the negative control group are 25 mg/kg. The test animals used in this experiment are six to eight weeks old C57BL/6 mice (BioLASCO Taiwan).

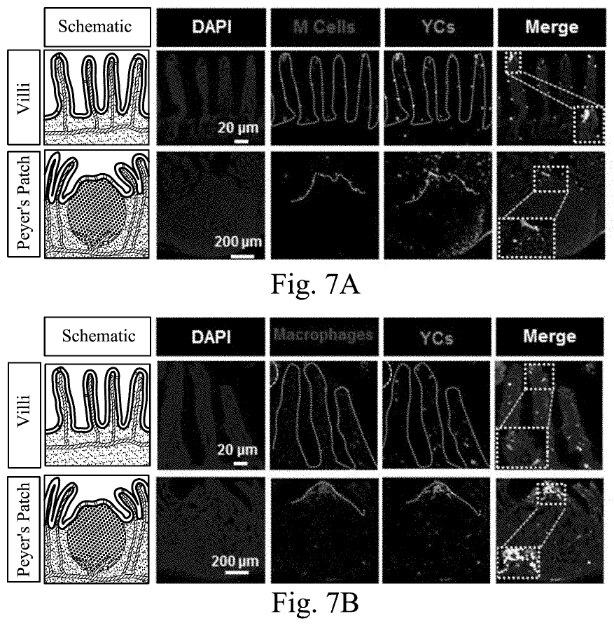

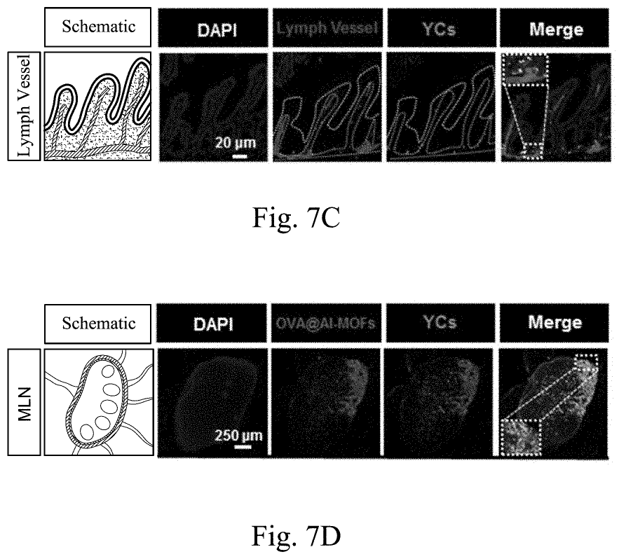

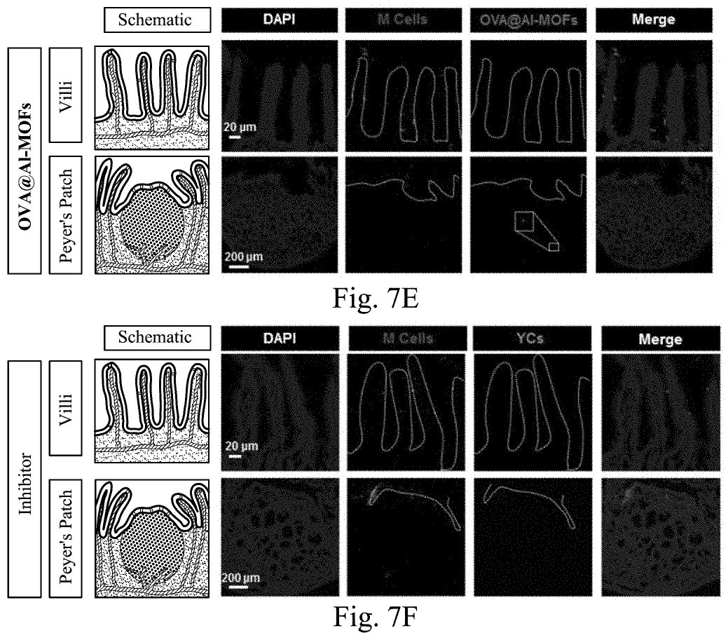

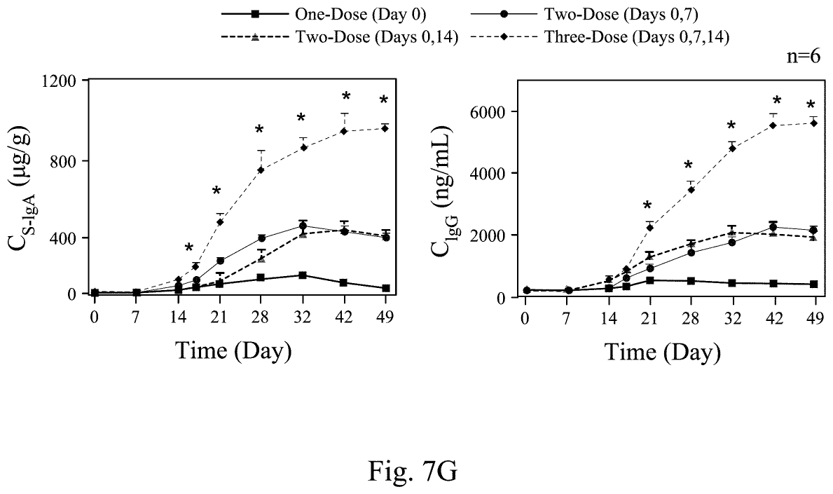

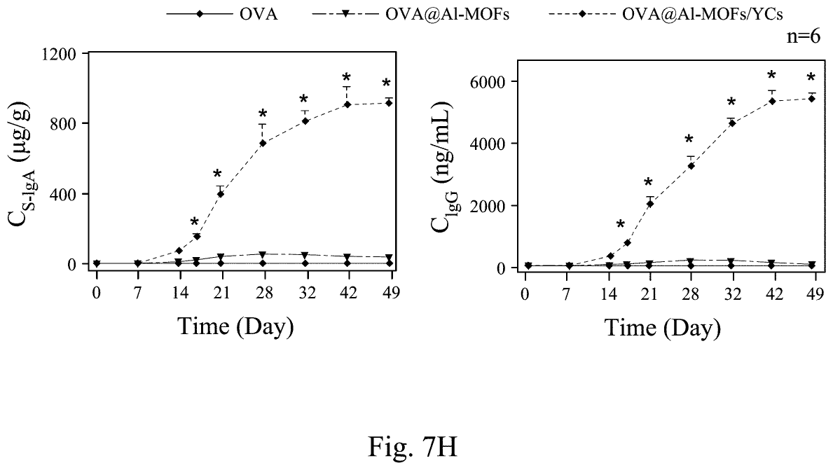

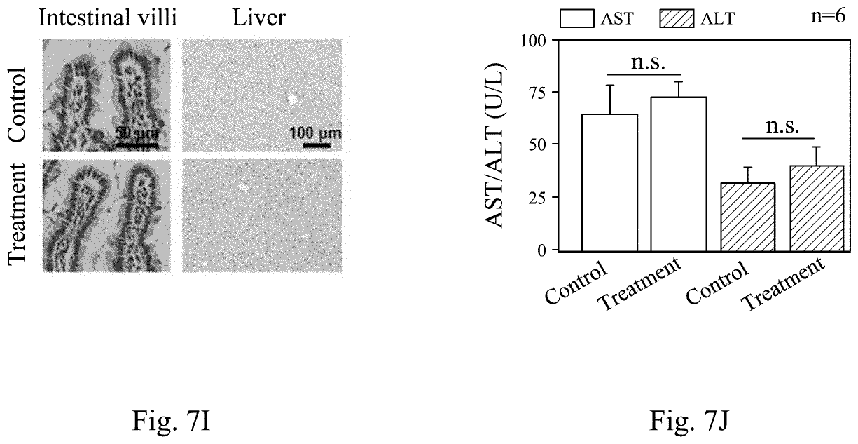

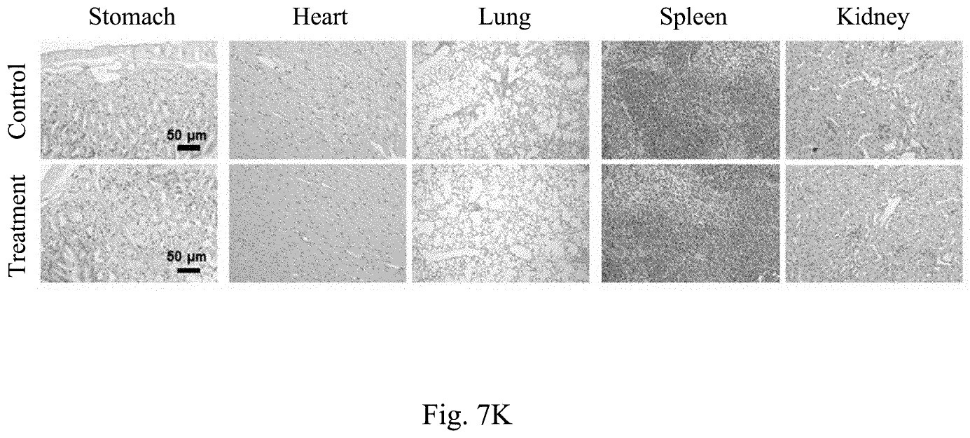

Please refer to FIGS. 7A, 7B, 7C, 7D, 7E and 7F, which show analytical results of in vivo transport route of the oral drug delivery system of Example 4 of the present disclosure. FIG. 7A shows schematic diagrams and CLSM images of M cells of the experimental group, FIG. 7B shows schematic diagrams and CLSM images of the macrophages in the intestine of the experimental group, FIG. 7C shows schematic diagrams and CLSM images of the lymphatic vessels of the experimental group, FIG. 7D shows schematic diagrams and CLSM images of the mesenteric lymph nodes (MLNs) of the experimental group, FIG. 7E shows schematic diagrams and CLSM images of the intestinal tract of the experimental control group, and FIG. 7F shows schematic diagrams and CLSM images of the intestinal tract of the negative control group.

In FIG. 7A, large numbers of the FITC-YCs (green) adhered/targeted M cells (red) enter the Peyer's patches. In FIGS. 7B to 7D, the FITC-YCs (green) can be endocytosed by macrophages, transported through mesenteric lymphatic vessels, and ultimately accumulated in the MLNs. The results indicate that the β-glucans of the YCs participate in the recognition of the receptor Dectin-1 on M cells; macrophages are abundant in the GI tract and its neighboring lymphoid tissues; and the MLNs provide important sites for activating immune responses.

In FIG. 7E, only a small number of the AF633-OVA@AI-MOFs (pink) entered the Peyer's patches in the experimental control group. In FIG. 7F, no significant fluorescent signal from the FITC-YCs is detected in the villi or Peyer's patches in laminarin-pretreated negative control group, suggesting that laminarin blocked intestinal lymphatic transport.

### 2.4. OVA-Specific Mucosal and Systemic Immune Responses by Oral Drug Delivery System

The ability of the OVA@AI-MOFs/YCs to generate antigen-specific S-IgA and IgG antibodies in vivo is studied in this experiment. Oral immunization with the OVA@AI-MOFs/YCs is carried out in the C57BL/6 mice using various prime-boost combinations on Days 0, 7, and/or 14. Each oral dose is 100 μg OVA. Fecal extracts and serum samples are collected after immunization, and their levels of OVA-specific S-IgA and IgG are measured, respectively, using ELISA over duration of seven weeks. The prime-boost combinations include one-dose OVA@AI-MOFs/YCs (Day 0), two-dose OVA@AI-MOFs/YCs (Days 0 and 7 or Days 0 and 14), or three-dose OVA@AI-MOFs/YCs (Days 0, 7, and 14). Control mice are given three doses of free OVA or the OVA@AI-MOFs on Days 0, 7, and 14 (n=6 in each group). Each dose contains the same amount of OVA (100 μg).

Please refer to FIG. 7G, which shows analytical results of concentration of OVA-specific S-IgA antibodies and IgG antibodies in test animals after administering with the oral drug delivery system of Example 4 of the present disclosure under various dosing regimens. In FIG. 7G, S-IgA and IgG titers in the C57BL/6 mice that had been vaccinated with one or two doses remain relatively low throughout the study, whereas those in the C57BL/6 mice that had been vaccinated with three doses steadily increase and strong mucosal and systemic immune responses are obtained (P<0.05).

The potencies of soluble OVA (free OVA), the OVA@AI-MOFs, and the OVA@AI-MOFs/YCs in eliciting immune responses using a three-dose oral immunization schedule are evaluated and compared. Please refer to FIG. 7H, which shows analytical results of concentration of OVA-specific S-IgA antibodies and IgG antibodies in the test animals after administering with free OVA, the OVA@AI-MOFs and the OVA@AI-MOFs/YCs using a three-dose oral immunization schedule. In FIG. 7H, considerable differences in the potencies of these vaccines are observed. The S-IgA and IgG titers that are stimulated by OVA alone or the OVA@AI-MOFs are negligible relative to those that are stimulated by the OVA@AI-MOFs/YCs (P<0.05). The low potency of soluble OVA may be caused by antigen degradation by proteolytic enzymes, while that of the OVA@AI-MOFs is attributable to the low dose of antigen that is orally absorbed. In contrast, the OVA@AI-MOFs/YCs delivery platform protected the antigen during transport in the GI tract and specifically targeted M cells, increasing the transepithelial absorption of the OVA@AI-MOFs/YCs, and facilitating their translocation by macrophages through the lymphatic system. The OVA@AI-MOFs/YCs ultimately accumulate in the MLNs, generating high concentrations of mucosal S-IgA and serum IgG antibodies.

### 2.5. In Vivo Toxicity of Oral Drug Delivery System

To measure the potential in vivo toxicity of OVA@AI-MOFs/YCs, the test mice, following a three-dose oral immunization schedule of the OVA@AI-MOFs/YCs, are sacrificed at the end of the experiment 2.4 (in week 7 post-oral administration), and their main organs (small intestine, liver, stomach, heart, lung, spleen, and kidney) are retrieved, fixed in 10% neutral buffered formalin, and stained with haematoxylin and eosin (H&E). Images of the H&E-stained tissue sections are captured using an IX83 inverted microscope (Olympus, Tokyo, Japan). To evaluate toxicity in the liver, the activities of aspartate aminotransferase (AST) and alanine aminotransferase (ALT) enzymes in serum are measured using a commercial kit (Thermo Fisher Scientific).

Please refer to FIGS. 7I, 7J and 7K. FIG. 7I shows histological photomicrographs of the intestinal villi and the liver sections of the test animals. FIG. 7J shows analytical results of AST and ALT enzyme levels in plasma of the test animals. FIG. 7K shows histological photomicrographs of stomach, heart, lung, spleen and kidney sections of the test animals. The results showed that compared with the normal control group, no evidence of an inflammatory reaction in any of the experimental tissues of the treatment groups treated with the OVA@AI-MOFs/YCs. Moreover, the AST and ALT enzyme blood levels that are obtained from the normal control group and the experimental groups are similar (P>0.05). Taken together, the above experimental results demonstrate that the orally administered the oral drug delivery system can function as a safe vehicle for delivering the biological macromolecule encapsulated therein to produce high levels of mucosal S-IgA and serum IgG antibodies and longer-lasting immunity on repeated immunizations.

### 2.6. Transport Route to Brain of Oral Drug Delivery System

The previous experiments have confirmed that the oral drug delivery system of the present disclosure can be endocytosed by the macrophages and enter the intestinal lymphatic system. Whether the oral drug delivery system of the present disclosure can further deliver the biological macromolecule encapsulated therein to the brain via the lymphatic system is studied in this experiment. The test animals in the treatment group are orally treated fluorescently labeled OVA@AI-MOFs/YCs, and the test animals are sacrificed 6 hours after oral administration. The distribution of the FITC-YCs in the brains, hearts, lungs, livers, spleens, pancreas and kidneys of the test animals are confirmed using an in vivo imaging system (IVIS). The brain tissue sections are retrieved for immunofluorescence staining, and observed with CLSM and photographed. The untreated test animals are as a control group.

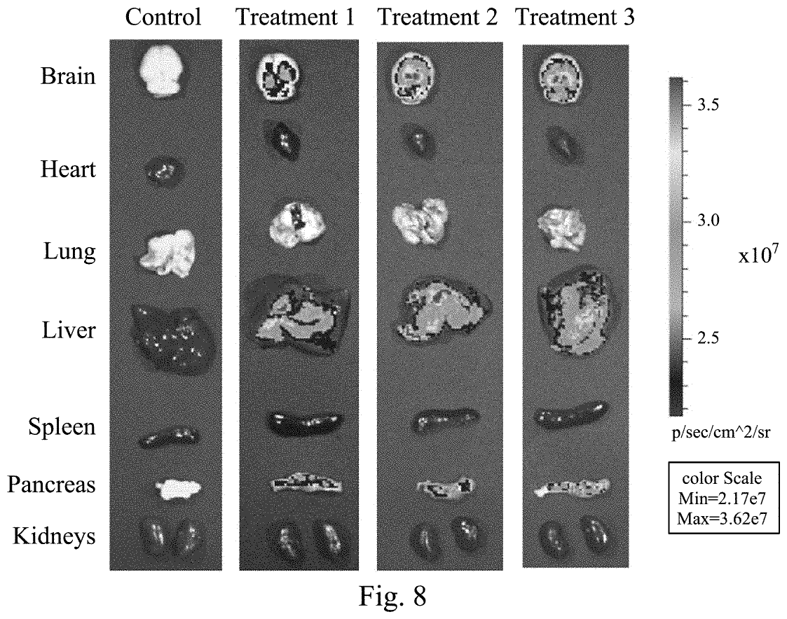

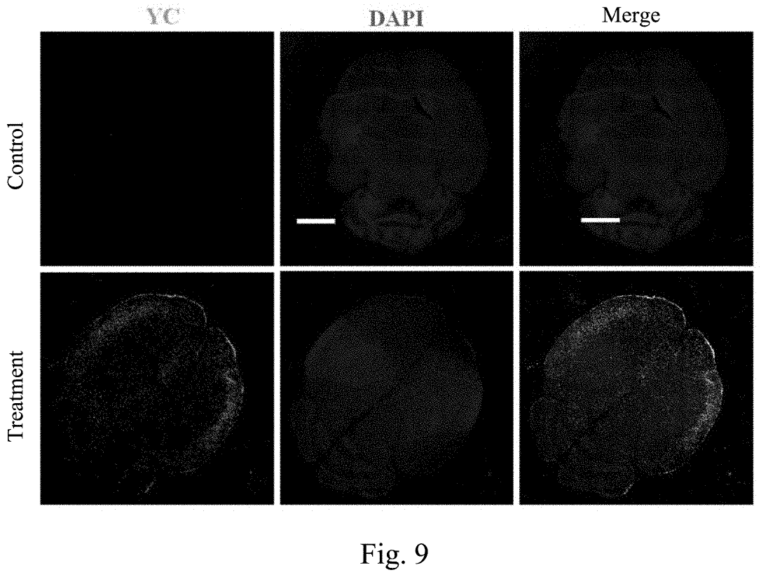

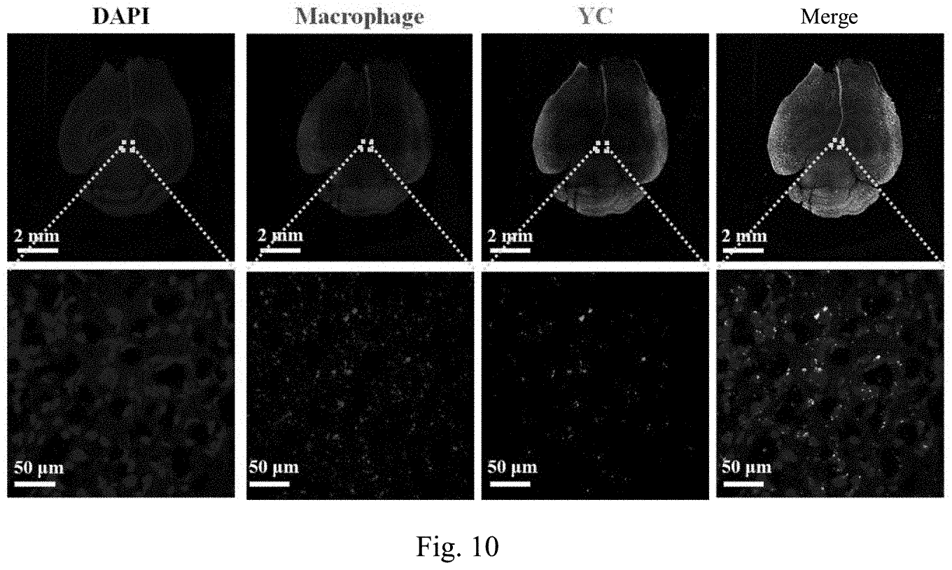

Please refer to FIGS. 8, 9 and 10. FIG. 8 shows analytical results of in vivo imaging system of the brain, heart, lungs, liver, spleen, pancreas and kidneys after administration with the oral drug delivery system of the present disclosure. FIG. 9 shows CLSM images of a brain tissue of the test animal administered with the oral drug delivery system of the present disclosure. FIG. 10 shows analytical results of immunofluorescence staining on the brain tissue of the test animal administered with the oral drug delivery system of the present disclosure. In FIGS. 8 and 9, the distribution of the FITC-YCs in the brain is detected in the treatment group orally treated with the OVA@AI-MOFs/YCs, while the FITC signal does not be detected in the control group. In FIG. 10, the intracellular colocalization of green fluorescence (FITC-YCs) and red fluorescence (macrophages) is clearly visible, indicating that the OVA@AI-MOFs/YCs can enter the brain through the lymphatic system after being endocytosed by macrophages. The results indicate that the oral drug delivery system of the present disclosure has the ability to deliver the biological macromolecules encapsulated therein to the brain.

In summary, the oral drug delivery system of the present disclosure can protect the biological macromolecules encapsulated therein by biomimetically mineralized metal organic framework for resisting highly acidic and degradative the GI conditions and keeping the activity of the biological macromolecule encapsulated therein, and can act synergistically as a delivery vehicle and the adjuvant. The YCs loaded with the biomimetic mineralized carrier can target M cells in the intestinal tract, increasing transepithelial absorption of the oral drug delivery system, followed by subsequent endocytosis in local macrophages, ultimately accumulating in the mesenteric lymph nodes, and yielding long-lasting immune response. Further, the oral drug delivery system of the present disclosure can deliver the biological macromolecules encapsulated therein to the brain through the lymphatic system after being endocytosed by macrophages. Therefore, the oral drug delivery system of the present disclosure can deliver brain drugs by oral administration.

The method for fabricating the oral drug delivery system is a simple one-pot method for fabricating the biomimetic mineralized carrier. The organic ligands and the metal ions are processed by mild ultrasound to synthesize a nanoscale metal organic framework, and further mimic the secretion of inorganic minerals by living organisms to form exoskeletons to encapsulate the biological macromolecules in the metal organic framework to form the biomimetic mineralized carrier with the positive charge on the surface. Furthermore, the biomimetic mineralized carrier is loaded into the yeast capsule with the negative charge on the surface by electrostatic force to form the oral drug delivery system.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An oral drug delivery system, comprising:
a biomimetic mineralized carrier having a surface with a positive charge, comprising:
a metal organic framework having an internal space, wherein a surface of the metal organic framework has a plurality of pores; and
a biological macromolecule encapsulated in the internal space of the metal organic framework; and
a yeast capsule composed of a β-glucan cell-wall shell that removes a cytoplasm from a yeast, wherein the yeast capsule has a surface with a negative charge, and the biomimetic mineralized carrier is loaded into the yeast capsule by an electrostatic force.

2. The oral drug delivery system of claim 1, wherein a particle size of the biomimetic mineralized carrier ranges from 25 nm to 100 nm.

3. The oral drug delivery system of claim 1, wherein the metal organic framework is MIL-53 (Al, Fe, Cr), MIL-100 (Al, Fe, Cr), MIL-101 (Al, Fe, Cr), MIL-127 (Al, Fe, Cr), PCN-88 (Cu), NU-1000 (Zr) or UIO-66 (Zr).

4. The oral drug delivery system of claim 1, wherein the biological macromolecule is a nucleic acid or a protein.

5. The oral drug delivery system of claim 4, wherein the nucleic acid is selected from the group consisting of an oligo-double-stranded DNA, a poly-double-stranded DNA, an oligo-single-stranded DNA, a poly-single-stranded DNA, an oligo-single-stranded RNA and a poly-single-stranded RNA.

6. The oral drug delivery system of claim 1, wherein the yeast is Saccharomyces cerevisiae, Candida albicans, Rhodotorula rubra or Torulopsis utilis.

7. A method for fabricating the oral drug delivery system of claim 1, comprising:
providing a mixture, wherein the mixture comprises an organic ligand, a metal ion, the biological macromolecule and water;
performing a coating step, wherein the mixture is subjected to a coordination reaction between the organic ligand and the metal ion in a sonication manner to form the internal space, and the biological macromolecule is in situ encapsulated in the internal space to form the biomimetic mineralized carrier having the surface with the positive charge;
collecting the biomimetic mineralized carrier;
providing a first solution comprising the biomimetic mineralized carrier;
providing a second solution comprising the yeast capsule, wherein the yeast capsule is composed of the β-glucan cell-wall shell that removes the cytoplasm from the yeast by a chemical method, and the yeast capsule has the surface with the negative charge; and
performing a loading step, wherein the first solution is mixed with the second solution and then shaken for a shaking time, and the biomimetic mineralized carrier is loaded into the yeast capsule by the electrostatic force to form the oral drug delivery system.

8. The method of claim 7, wherein a concentration ratio of the organic ligand, the metal ion and the biological macromolecule in the mixture is 1:1:0.004 to 1:1:0.018.

9. The method of claim 7, wherein the organic ligand is 2-amino terephthalic acid, terephthalic acid, 3,3′-(naphthalene-2,7-diyl) dibenzoic acid, 3,3′,5,5′-azobenzenetetracarboxylic acid or biphenyl-4,4′-dicarboxylic acid.

10. The method of claim 7, wherein the metal ion is formed by dissolving a metal salt in hydrolysis, and the metal salt is AlCl₃, Al₂(SO₄)₃, Al(NO₃)₃, aluminium isopropoxide, FeCl₃, Fe₂(SO₄)₃, Fe(NO₃)₃, CuCl₂, CuSO₄, Cu(NO₃)₂, ZrCl₄, Zr(NO₃)₄, Zr(SO₄)₂, CrCl₃, Cr(NO₃)₃ or zirconium citrate.

11. The method of claim 7, wherein the biological macromolecule is a nucleic acid or a protein.

12. The method of claim 11, wherein the nucleic acid is selected from the group consisting of an oligo-double-stranded DNA, a poly-double-stranded DNA, an oligo-single-stranded DNA, a poly-single-stranded DNA, an oligo-single-stranded RNA and a poly-single-stranded RNA.

13. The method of claim 7, wherein in the loading step, a weight ratio of the biomimetic mineralized carrier in the first solution and the yeast capsule in the second solution is 1:1 to 2:1.

14. The method of claim 7, wherein the sonication manner is to process the mixture using a sonicator at 30% to 50% amplitude at 0° C. for 90 to 150 minutes.

15. The method of claim 7, wherein the shaking time in the loading step is 2 to 6 hours.
